(19) United States
(12) Patent Application Publication
(10) Pub. No.: US 2016/0361253 A1
(43) Pub. Date: Dec. 15, 2016

(54) Novel Methods for Delivering Therapeutic Agents to the Eye via Nasal Passages

(71) Applicant: Noveome Biotherapeutics, Inc., Pittsburgh, PA (US)

(72) Inventor: Larry R. Brown, Newton, MA (US)

(73) Assignee: Noveome Biotherapeutics, Inc., Pittsburgh, PA (US)

(21) Appl. No.: 15/180,855

(22) Filed: Jun. 13, 2016

Related U.S. Application Data
(60) Provisional application No. 62/175,239, filed on Jun. 13, 2015.

(51) Int. Cl. A61K 9/00; A61K 35/50; A61K 38/19

(52) U.S. Cl. CPC A61K 9/0043; A61K 38/19; A61K 35/50

(57) ABSTRACT

The invention is directed to delivering therapeutic agents to the eye for the purpose of treating ophthalmic disorders, diseases and injuries. In particular, the invention is directed to delivering therapeutic agents to the eye for the purpose of treating ophthalmic disorders, diseases and injuries by targeted intranasal administration of the therapeutic agents. The invention is specifically directed to treating disorders, diseases and injuries of the cornea and ocular surface, treating retinal disorders, diseases and injuries and optic nerve disorders, diseases and injuries by targeted intranasal administration of the therapeutic agents.

### FIELD OF THE INVENTION

The field of the invention is directed to delivering therapeutic agents to the eye for the purpose of treating ophthalmic disorders, diseases and injuries. In particular, the field of the invention is directed to delivering therapeutic agents to the eye for the purpose of treating ophthalmic disorders, diseases and injuries by targeted intranasal administration of the therapeutic agents. The field of the invention is specifically directed to treating disorders, diseases and injuries of the cornea and ocular surface, treating retinal disorders, diseases and injuries and optic nerve disorders, diseases and injuries by targeted intranasal administration of the therapeutic agents.

### DESCRIPTION OF RELATED ART

A PhD thesis by Sandra R. Alcalá, July 2009, entitled “Investigation of the Intranasal Delivery Method as a Means of Targeting Therapeutic Agents to the Injured Retina and Optic Nerve” studied intranasal delivery of ciliary neurotrophic factor (CNTF) to understand and treat ischemic optic neuropathy.

Wong, Y. and Zuo, Z., describe brain disposition and catalepsy after intranasal delivery of loxapine: role of metabolism in PK/PD of intranasal CNS drugs (Pharm Res 30(9):2368-2384, (2013).

Thorne R G, Hanson L R, Ross T M, Tung D, Frey W H 2^{nd} describe delivery of interferon-beta to the monkey nervous system following intranasal administration (Neuroscience, Mar 27;152(3):785-97, doi: 10.1016/j.neuroscience.2008.01.013. Epub 2008, Jan 16 2008).

Renner D B, Svitak A L, Gallus N J, Ericson M E, Frey W H 2nd, Hanson L R describe intranasal delivery of insulin via the olfactory nerve pathway (J Pharm Pharmacol doi: 10.1111/j.2042-7158.2012.01555.x., 1709-1714, 2012).

Hanson, L R et al. describe intranasal administration of CNS therapeutics to awake mice (J Vis Exp 74(e4440):1-7, 2013).

Bitter, C. et al. review various consideration for nasal drug delivery in humans (Surber C., Elsner P., Farage, M A (eds): Topical Applications and the Mucosa. Curr Probl Dermatol. Basel, Karger, 2001, vol. 40, pp 20-35).

Hoekman and Ho (AAPS PharmSciTech, Vol. 12, No. 2, 2011, pp. 534-542) describe the effects of localized hydrophilic mannitol and hydrophobic nelfinavir administration targeted to olfactory epithelium on brain distribution and reported that targeted intranasal delivery to deliver agents to the brain is superior to non-targeted intranasal delivery.

### BACKGROUND OF THE INVENTION

There are several major ophthalmological disorders, diseases and injuries that affect the cornea, lens, retina and optic nerve. Serious corneal disorders, diseases and injuries include corneal ulcers, corneal wounds (i.e., thermal, chemical, physical, surgical), keratitis (inflammation of the cornea), allergic conjunctivitis, dry eye syndrome, and Sjogren's syndrome. Serious lens disorders include cataracts and refractive errors. The most serious disorders and diseases of the retina include macular holes, retinal degeneration, diabetic retinopathy, retinal ischemia, diabetic macular edema, wet and dry macular degeneration, glaucoma, Retinitis Pigmentosa, Usher syndrome, Stargardt disease, retinal detachment, choroideremia, and retinoschisis. Serious diseases of the optic nerve include optic neuritis and neuromyelitis optica. In addition, ophthalmic diseases such as glaucoma, which is characterized by ocular hypertension, can cause damage to the optic nerve.

The cornea is the transparent front part of the eye that covers the iris, pupil, and anterior chamber. The human cornea has five layers. From the anterior to posterior the five layers of the human cornea are the 1) corneal epithelium, a thin layer of stratified squamous epithelial cells which are fast-growing and easily-regenerated cells that are kept moist with tears. The corneal epithelium is continuous with the conjunctival epithelium which is composed of about 6 layers of cells which are shed constantly and are regenerated by cell division in the basal layer; 2) Bowman's layer which is a tough layer of condensed collagen fibers that protects the corneal stroma, which consists of similar irregularly arranged collagen fibers; 3) The corneal stroma which is a thick, transparent middle layer, consisting of regularly arranged collagen fibers along with sparsely distributed interconnected keratocytes, which are the cells for general repair and maintenance; 4) Descemet's membrane which is a thin acellular layer that serves as the modified basement membrane of the corneal endothelium, from which the cells are derived; and 5) the corneal endothelium which is a simple squamous or low cuboidal monolayer of mitochondria-rich cells responsible for regulating fluid and solute transport between the aqueous and corneal stromal compartments.

The lens is a transparent, biconvex structure in the eye that, along with the cornea, helps to refract light to be focused on the retina. The lens has three main parts: the lens capsule, the lens epithelium, and the lens fibers. The lens capsule forms the outermost layer of the lens and the lens fibers form the bulk of the interior of the lens. The cells of the lens epithelium, located between the lens capsule and the outermost layer of lens fibers, are found only on the anterior side of the lens.

The retina is a very thin layer of light-sensitive neural tissue lining at the inner posterior surface of the eyeball. It is composed of six classes of neurons and one type of glial cell that are interconnected in a highly organized structure. The rod and cone photoreceptor cells reside in the outer nuclear layer; the horizontal, bipolar, and amacrine interneurons plus the Müller glial cells reside in the inner nuclear layer; and the retinal ganglion cells and displaced amacrine cells reside in the ganglion cell layer. The major function of the retina is to convert light signals detected by photoreceptor cells into electrical impulses, which are then transmitted to the brain via the optic nerve which is derived from the projecting axons of the retinal ganglion cells. Any loss and/or damage of the various retinal cell types will result in disruption of the normal transmission of nerve impulses and lead to impaired vision.

The optic nerve, also known as cranial nerve II, is a paired nerve that transmits the visual information from the retina to the brain. The optic nerve is derived from optic stalks during the seventh week of fetal development and is composed of retinal ganglion cell axons and glial cells. In humans, the optic nerve extends from the optic disc to the optic chiasm and then continues as the optic tract to the lateral geniculate nucleus, pretectal nuclei, and superior colliculi in the brain. The fibers of the optic nerve are covered with myelin produced by oligodendrocytes, rather than Schwann cells of the peripheral nervous system, and are encased within the meninges.

Many ophthalmic disorders, diseases and injuries are treated with surgery (i.e., cataracts). In some instances, treatment has focused on gene therapy to correct inheritable disorders such as those found in Retinitis Pigmentosa. Other areas of treatment and current research are directed towards evaluating the role of growth factors and/or cytokines. In some instances, the growth factors and/or cytokines have been evaluated for their ability to prevent or protect against retinal cell death or for generating new retinal cells to replace lost ones. Similar studies with growth factors and/or cytokines aim to protect and/or regenerate limbal stem cells to treat corneal injuries such as corneal wounds. Still other treatment approaches use various inhibitors of neovascularization (i.e., VEGF inhibitors such as EYLEA®) to prevent or reduce the amount of new blood vessel growth in the eye and associated vascular leak and hemorrhage such as that seen in diabetic retinopathy and age-related macular degeneration. Other treatment options for corneal disorders/diseases/injuries include antibiotics, antifungals or antivirals if infection is present; mitomycin C; topical steroids to treat inflammation; bandage contact lens; fibrin glue; tarsorraphy (partial suturing of the eyelids); autologous serum; Gunderson flap; and corneal transplant.

Another area of research is directed to evaluating the potential of stem cells to replace damaged or lost retinal cells or corneal epithelial cells, including limbal stem cells (see, for example, Chacko, D. M., et al, (Biochem Biophy Res Commun 2000, 268(3):842-6); Otani, A., et al, (J Clin Invest 2004 114(6):765-7); Smith, L. E. (J Clin Invest 2004 114(6):755-7; Ahmed, S., et al, (Stem Cells, 2007, Jan 25 e-publication). Also being studied is retinal transplantation (see Ng, T. F., et al, Chem Immunol Allergy, 2007, 92:300-16).

Additional treatment options include topically delivering therapeutic agents to the surface of the eye or injecting therapeutic agents into the vitreous of the eye. For most patients, injections into the eye are unpleasant and uncomfortable. Therefore, it is an object of the instant invention to provide a treatment option for patients suffering from ophthalmic disorders, diseases and injuries, in particular, corneal, lens, retinal, and optic nerve disorders, diseases and injuries, which encompass delivering the therapeutic agent non-invasively to the ocular tissues by targeted intranasal administration of a therapeutic agent. Such targeted intranasal administration would be particularly desirable in patients who currently require injections into the vitreous for treatment of their ophthalmic condition or those wherein systemic administration is not possible because the therapeutic agent cannot cross the blood-brain barrier.

### BRIEF SUMMARY OF THE INVENTION

Applicant has discovered that when a therapeutic agent is administered by targeted intranasal delivery to a specific region in the nasal cavity, the agent can be found in the optic nerve, optic chiasm, the optic nerve head, the eye choroid, the retinal pigment epithelium, the retina and the eye vitreous humor. Applicant has also discovered that Amnion-derived Cellular Cytokine Solution (ACCS) (for details see U.S. Pat. Nos. 8,058,066 and 8,088,732, both of which are incorporated herein by reference), now termed ST266, exhibits anti-inflammatory properties, anti-vascular permeability properties, myelin sheath protective properties, neuroprotective properties, and wound healing properties. Amnion-derived Multipotent Progenitor (AMP) cell compositions, from which ST266 is derived (for details see U.S. Pat. Nos. 8,058,066 and 8,088,732, both of which are incorporated herein by reference) also exhibit many of these properties. Applicant has also developed novel cells called AMP-N cells which produce a novel secretome called ACCS-N (see U.S. Publication No. 2015-0196603-A1, published on Jul. 16, 2015 and incorporated herein by reference) both of which are suitable for use in practicing the methods of the invention. As described herein, Applicant has discovered that ST266 and/or AMP cells, and/or ACCS-N and/or AMP-N cells when administered by targeted intranasal delivery, for example as a liquid or a fine powder nasal spray, provide an effective means of treating ophthalmic disorders, disease and injuries. This is because the compositions are specifically targeted to the nasal mucosa which is adjacent to the foramina of the cribriform plate located at the superior aspect of the nasal cavity such that the ST266 and/or AMP cells, and/or ACCS-N and/or AMP-N cell compositions can permeate through the foramina into the cranial cavity at the location of the optic nerve and globe of the eye.

In accordance with Applicant's invention, any therapeutic agents, including those described herein, as well as second generation versions of the disclosed compositions and functional equivalents thereof, that are useful for treating ophthalmic conditions are suitable for use in the methods of the invention. The only requirement is that the agent be able to be formulated for targeted intranasal administration. Therefore, both small and large molecular agents can be used, including complex biological compositions such as ST266 and ACCS-N, and cells such as AMP cells and AMP-N cells describe herein.

While general intranasal delivery of therapeutics agents is common, it is important to note that targeted intranasal delivery is different from and superior to non-targeted intranasal delivery. For example, Hoekman and Ho (AAPS PharmSciTech, Vol. 12, No. 2, 2011, pp. 534-542) reported that targeted intranasal delivery to deliver agents to the brain is superior to non-targeted intranasal delivery. Non-targeted intranasal delivery is not suitable for practicing the methods of the invention. This is because non-targeted intranasal delivery such as that accomplished by simply spraying an agent into the nostrils by squeezing a plastic bottle merely deposits the agent on the mucosa of the nasal cavity. The agent then, at best, crosses the nasal epithelium and is picked up by local capillaries for systemic distribution before it can be cleared away by the normal clearing mechanisms present in the nasal cavity such as mucociliary action (see Bitter, et al., Surber C., Elsner P., Farage, M A (eds): Topical Applications and the Mucosa. Curr Probl Dermatol. Basel, Karger, 2001, vol. 40, pp 20-35). Thus to be effective, non-targeted intranasal administration requires a combination of suitable permeability of the agent across the nasal epithelium to achieve a therapeutic systemic dose and a suitable resident time for the agent on the mucosa (see Bitter, et al. Surber C., Elsner P., Farage, M A (eds): Topical Applications and the Mucosa. Curr Probl Dermatol. Basel, Karger, 2001, vol. 40, pp 20-35).

As will be describe in detail below in the Examples section, rodent models were used to demonstrate the efficacy of ST266. In these models, the ST266 was administered to the animal by dripping the ST266 into the nostrils with a pipette while the animals were in a supine position such that the ST2666 could flow to the superior aspect of the nasal cavity. However, laying human patients on their backs and dripping a drug in their nose with the hope that amounts sufficient to produce a therapeutic effect will reach their target is neither practical nor acceptable medical practice. Rather, being able to administer a specific dose to a specific area of the nasal cavity is desirable. To address this issue, non-human primate studies (described below in the Examples section) were conducted to establish that targeted intranasal delivery of an agent, in this case Evans blue dye or I-¹²⁵ radiolabeled ST266, to the optic nerve, the optic chiasm, and globe of the eye, the caudate putamen, the cerebellum, the entorhinal cortex, the prefrontal cortex, the hippocampus, the olfactory bulb, the olfactory nerve, the substantia nigra, the trigeminal nerve, the trochlear nerve, could be successfully accomplished.

Furthermore, due to the presence of the blood-brain barrier, systemic distribution of therapeutic agents to the central nervous system and other organs and tissues protected by the blood-brain barrier, such as the optic nerve and other ocular tissues, is generally ineffective. Some studies have demonstrated that certain agents can be administered intranasally and be deposited in certain brain regions. However, surprisingly, Applicant has shown that by specifically targeting the nasal mucosa which is adjacent to the foramina of the cribriform plate located at the superior aspect of the nasal cavity, therapeutic agents can permeate through the foramina into the cranial cavity at the location of the optic nerve and globe of the eye. Accordingly, they are delivered directly to the ocular tissues where needed without having to cross the blood-brain barrier or travel systemically. Also surprisingly, even large molecular weight molecules such as proteins are able to be deposited in ocular tissues by this targeted route of administration, including complex mixtures of large molecular weight biomolecules such as those contained in ST266 and ACCS-N. This discovery represents a significant improvement in how physicians may be able to treat ocular diseases, especially back of the eye diseases (meaning diseases affecting eye structures that are not the anterior surface and related structures), because of the ease and non-invasiveness of the procedure. The benefits to patients is that they may no longer have to endure multiple intraocular injections to treat such diseases, they will be able to self-administer the therapeutic agent(s) and, even more importantly, they may have treatment options for diseases that heretofore could not be treated because there was no suitable delivery route.

Thus it is an object of the instant invention to provide novel methods for treating ophthalmic diseases, disorders and injuries including corneal, intravitreal, retinal, and optic nerve disorders, diseases and injuries by targeted intranasal administration of therapeutic agents. Such novel methods for treating ophthalmic disorders/diseases/injuries by targeted intranasal administration of therapeutic agents utilize novel compositions including ST266 and/or AMP cells, and/or ACCS-N and/or AMP-N cell compositions, each alone and/or in combination with each other and/or with other agents including active and/or inactive agents.

Accordingly, a first aspect of the invention is a method for delivering a therapeutic agent to the eye in a patient in need thereof comprising targeted intranasal administration of the therapeutic agent to the patient. In one embodiment the therapeutic agent is targeted to the superior aspect of the nasal cavity which is adjacent to the cribriform plate. In another embodiment a device is used to effect the targeted intranasal administration of the therapeutic agent. In still another embodiment the patient is in an upright position while using the device to effect the targeted intranasal administration.

In one embodiment the therapeutic agent is a small molecular weight agent. In another embodiment the small molecular weight agent is a biological. In another embodiment the small molecular weight agent is a chemical. In yet another embodiment the small molecular weight agent has a molecular weight equal to or less than 900 daltons. In another particular embodiment, the small molecular weight agent is water-soluble. In another particular embodiment, the small molecular weight agent is amphiphilic.

Another embodiment is one in which the therapeutic agent is a large molecular weight agent. In another embodiment the large molecular weight agent is a biological. In another embodiment the large molecular weight agent is a chemical. In still another embodiment the large molecular weight agent has a molecular weight greater than 900 daltons.

Another embodiment is one in which the therapeutic agent is a complex biological composition comprised of numerous biological molecules. In a specific embodiment the complex biological composition comprised of numerous biological molecules is selected from the group consisting of ST266 and ACCS-N.

In still another embodiment the therapeutic agent is a population of cells. In a specific embodiment the population of cells is selected from the group consisting of AMP cells and AMP-N cells.

In yet another embodiment the patient is afflicted with an ophthalmic disorder, disease or injury. In a specific embodiment wherein the ophthalmic disorder, disease or injury is selected from the group consisting of a corneal disorder, disease or injury, a lens disorder, disease or injury, a retinal disorder, disease or injury and an optic nerve disorder, disease or injury.

In still another embodiment, the therapeutic agent is administered in combination with other agents or treatment modalities. In a particular embodiment the other agents are active agents. And in a specific embodiment the active agents are selected from the group consisting of growth factors, cytokines, inhibitors, immunosuppressive agents, steroids, chemokines, antibodies, antibiotics, antifungals, antivirals, mitomycin C, and other cell types. In another particular embodiment the inhibitor is a LINGO inhibitor. LINGO is a protein found in nerve cells and myelin-making oligodendrocyte cells. In another particular embodiment the inhibitor is Glatiramer (TEVA Pharmaceuticals). LINGO is a protein found in nerve cells and myelin-making oligodendrocyte cells. In another particular embodiment the inhibitor is a VEGF inhibitor. Examples of VEGF inhibitors include Eylea® (Regeneron Pharmaceuticals, Inc.), Macugen® (EyeTech Pharmaceuticals), Avastin® (Genentech) and Lucentis® (Genentech). In another particular embodiment, the immunosuppressive agents are cyclosporine, methotrexate, FK-506 and corticosteroids. In another particular embodiment, the other cell types are retinal progenitor cells (see, for example, Coles, B. L., et al., PNAS USA 2004, 101(44):15772-7.). In another particular embodiment, the other treatment modalities are selected from the group consisting of bandage contact lens, fibrin glue, tarsorraphy (partial suturing of the eyelids), autologous serum, Gunderson flap and corneal transplant.

In specific embodiments the corneal disorder, disease or injury is keratitis, corneal ulcers, corneal wounds, dry eye syndrome, Sjogren's syndrome, allergic conjunctivitis, and corneal transplantation; the corneal wounds are selected from the group consisting of chemical wounds, thermal wounds, surgical wounds and mechanical wounds; the keratitis is caused by amoebic, bacterial, fungal or viral infection; photokeratitis; exposure (eyelid dysfunction); chemical injury; trauma; surgery; keratoconus; Fuchs' dystrophy; or keratoconjunctivitis sicca; and the surgery is selected from the group consisting of laser-assisted in situ keratomileusis (LASIK), photorefractive keratectomy (PRK), cataract, corneal transplant and pterygium surgery.

In another specific embodiment, the retinal disorder, disease or injury is macular holes, retinal detachment, retinal degeneration, retinitis pigmentosa (RP), light-induced retinal degeneration, choroideremia, retinoschisis, diabetic retinopathy, retinal ischemia, retinopathy of prematurity (ROP), and retinal transplantation.

In another embodiment the optic nerve disorder, disease or injury is optic neuritis, optic neuropathy, non-arteritic anterior ischemic optic neuropathy (NAION), arteritic anterior ischemic optic neuropathy (AION), traumatic optic neuropathy (TON), Leber's optic neuropathy (LHON) or Leber optic atrophy, dominant optic atrophy, or dominant optic atrophy, Kjer's type, recessive optic atrophy, radiation-induced optic neuropathy (RION), neuromyelitis optica spectrum disorder (NMOSD), optic nerve crush, optic nerve blunt force trauma, and glaucoma.

In another embodiment the other treatment modalities are selected from the group consisting of bandage contact lens, fibrin glue, tarsorraphy (partial suturing of the eyelids), autologous serum, Gunderson flap and corneal transplant.

In still another embodiment the therapeutic agent is formulated for targeted intranasal administration. In a specific embodiment the targeted intranasal administration is aerosol or spray administration. In yet another embodiment the therapeutic agent is formulated as a lyophilized dry powder nasal formulation.

Other features and advantages of the invention will be apparent from the accompanying description, examples and the claims. The contents of all references, pending patent applications and issued patents, cited throughout this application are hereby expressly incorporated by reference herein in their entirety. In case of conflict, the present specification, including definitions, will control.

### Definitions

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e., separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cells” or “EE cells” means a population of cells derived from the extraembryonic tissue.

As used herein, the term “Amnion-derived Multipotent Progenitor cell” or “AMP cell” means a specific population of epithelial cells derived from the amnion which have the characteristic of secreting VEGF, Angiogenin, PDGF and TGFβ2 and the MMP inhibitors TIMP-1 and/or TIMP-2 at physiologically relevant levels in a physiologically relevant temporal manner into the extracellular space or into the surrounding culture media. AMP cells have not been cultured in the presence of any non-human animal materials, making them and cell products derived from them suitable for human clinical use as they are not xeno-contaminated.

In addition to the characteristics described above, AMP cells have the following characteristics. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected and cultured under proprietary conditions, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved and novel compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of amnion epithelial cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of amnion epithelial cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e., diluted 1:5) and placed into a new tissue culture vessel to allow for their continued growth and viability. As used herein, “primary culture” means a freshly isolated, non-passaged cell population.

As used herein, the term “differentiation” means the process by which cells become progressively more specialized.

As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium.

As used herein, the term “ST266” means a novel conditioned medium that has been derived from AMP cells that have been cultured in basal media supplemented with human serum albumin under proprietary condition. ST266 has previously been referred to as “Amnion-derived Cellular Cytokine Solution” or “ACCS” and “amnion-derived cellular cytokine suspension” (for details see U.S. Pat. Nos. 8,058,066 and 8,088,732, both of which are incorporated herein by reference.

As used herein, the term “ACCS-N” means a novel conditioned medium that has been derived from AMP-N cells. “AMP-N” cells are a novel population of cells having certain, but not all, characteristics of neurons. ACCS-N and AMP-N cells are described in detail in U.S. Publication No. 2015-0196603-A1, published on Jul. 16, 2015, and incorporated herein in its entirety.

The term “physiological level” as used herein means the level that a substance in a living system is found and that is relevant to the proper functioning of a biochemical and/or biological process.

As used herein, the term “pooled” means a plurality of compositions that have been combined to create a new composition having more constant or consistent characteristics as compared to the non-pooled compositions.

The term “therapeutically effective amount” means that amount of a therapeutic agent necessary to achieve a desired physiological effect (i.e., treat an ophthalmic disorder, disease or injury).

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases. In some instances, it may be desirable to lyse the cells and retain the cellular membrane portion and discard the remaining portion of the lysed cells, or to retain both portions separately.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells, including a cell suspension or cells incorporated into a matrix or tissue, that are either in an undifferentiated, partially differentiated, or fully differentiated form into a human or other animal.

As used herein, the terms “a” or “an” means one or more; at least one.

As used herein, the term “adjunctive” means jointly, together with, in addition to, in conjunction with, and the like.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral and intrasternal injection or infusion.

The term “targeted intranasal” or “targeted intranasal delivery” or “targeted intranasal administration” as used herein means targeted delivery within the nasal structures at a precise location.

As used herein, the term “aerosol” means a cloud of solid or liquid particles in a gaseous medium.

The terms “particles”, “aerosolized particles”, and “aerosolized particles of formulation” are used interchangeably herein and shall mean particles of formulation comprised of any pharmaceutically active ingredient, optionally in combination with a carrier, (e.g., a pharmaceutically active drug and carrier). The particles have a size which is sufficiently small such that when the particles are formed they remain suspended in the air or gas for a sufficient amount of time such that a patient can deliver the particles by targeted intranasal administration.

As used herein, the term “nebulizer” means a device used to reduce a liquid medication to extremely fine cloudlike particles (i.e., an aerosol). A nebulizer may be useful in targeted intranasal delivery of a medication to a specific region of the nasal cavity if it is designed appropriately to accomplish targeted administration. Nebulizers may also be referred to as atomizers and vaporizers.

As used herein, the term “targeted intranasal delivery device” means a device that is capable of delivering a therapeutic agent to a precise location within the nasal cavity. Examples include the SipNose Ltd. (Yokneam Israel) nasal delivery systems as described in U.S. Pat. Nos. 9,339,617 and 9,227,031 and U.S. Published Application No. US-20160106937-A1, The Impel NeuroPharma (Seattle, Wash.) POD nasal delivery devices, and the Optinose US Inc. (Yardley, Pa.) nasal delivery devices.

The term “immediate-release” as used herein means that all of the pharmaceutical agent(s) is released into solution and into the biological orifice or blood or cavity etc. at the same time.

The term “targeted-release” or “targeted delivery” as used herein means that the pharmaceutical agent is targeted to a specific body region, tissue, biological orifice, tumor site or cavity, etc.

The terms “sustained-release”, “extended-release”, “time-release”, “controlled-release”, or “continuous-release” as used herein means an agent, typically a therapeutic agent or drug, that is formulated to dissolve slowly and be released over time.

As used herein the term “lyophilization” or “lyophilized” or “lyophilized powder” means a dehydration process typically used to preserve a perishable material or make the material more convenient for transport. Lyophilization works by freezing the material and then reducing the surrounding pressure to allow the frozen water in the material to sublimate directly from the solid phase to the gas phase. Other terms meaning lyophilization include freeze-drying and cryodesiccation.

As used herein, the term “co-administer” can include simultaneous or sequential administration of two or more agents, either by the same route of administration or by different routes of administration.

As used herein, the term “neurodegeneration” means the progressive loss of neurons in the nervous system. This includes but is not limited to immediate loss of neurons due to injury or disease followed by subsequent loss of connecting or adjacent neurons. One non-limiting example of neurodegeneration is retinal degeneration, in which the cells of the retina (i.e., photoreceptors known as rods and cones) are progressively lost.

As used herein, the term “neuroprotection” means to arrest and/or reverse progression of neurodegeneration following a nervous system injury or as a result of disease.

“Treatment” “treat” or “treating” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The term “ophthalmically acceptable” with respect to a formulation, composition or ingredient as used herein means having no persistent effect that is substantially detrimental to the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

As used herein the term “front of the eye” refers to the anterior surface of the eye and all related structures.

As used herein the term “back of the eye” refers to all eye structures that are not the anterior surface and related structures.

As used herein the term “standard animal model” refers to any art-accepted animal model in which the compositions of the invention exhibit efficacy.

### DETAILED DESCRIPTION

In accordance with the present invention there may be employed conventional molecular biology, microbiology, and recombinant DNA techniques within the skill of the art. Such techniques are explained fully in the literature. See, e.g., Green et al, 2012, “Molecular Cloning: A laboratory Manual”, Ausubel ed., 2016, “Current protocols in Molecular Biology”, Surzycki et al, 2000, “Basic Techniques in Molecular Biology” Park et al, 2011, “PCR Protocols”, Grandi et al, 2006, “In Vitro Transcription and Translation Protocols”, Anderson ed., 1999, “Nucleic Acid Hybridization”, Alberts et al, 2014, “Molecular Biology of the Cell”, Krebs et al, 2014, “Lewin's Genes XI”, Watson et al, 2014, “Molecular Biology of the Gene”, Nelson et al, 2013, “Lehninger Principles of Biochemistry”, Bonifacino ed., 2016, “Current Protocols in Cell Biology”, Mitry et al, 2012, “Human Cell Culture Protocols”, Helgason et al, 2011, “Basic Cell Culture Protocols”, Guisan et al, 2006, “Immobilization of Enzymes and Cells”, Owen et al, 2012, “Kuby Immunology”, Abbas et al, 2014, “Cellular and Molecular Immunology”′ Coligan ed., 2016, “Current Protocols in Immunology”.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Obtaining and Culturing of Cells

AMP cells—Various methods for isolating cells from the extraembryonic tissue, which may then be used to produce the AMP cells of the instant invention are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179).

Identifying AMP cells—Once extraembryonic tissue is isolated, it is necessary to identify which cells in the tissue have the characteristics associated with AMP cells (see definition above). For example, cells are assayed for their ability to secrete VEGF, Angiogenin, PDGF and TGFβ2 and the MMP inhibitors TIMP-1 and/or TIMP-2 into the extracellular space or into surrounding culture media. In some instances, it may be difficult or impossible to detect certain factors using standard assays. This may be because certain factors are secreted by the cells at physiological levels that are below the level of detection by the assay methods. It may also be that the factor(s) is being utilized by the AMP cells and/or by other local cells, thus preventing accumulation at detectable levels using standard assays. It is also possible that the temporal manner in which the factors are secreted may not coincide with the timing of sampling.

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e., human serum albumin) and no non-human animal protein; d) selecting AMP cells from the epithelial cell culture, and optionally e) further proliferation of the cells, optionally using additional additives and/or growth factors (i.e., recombinant human EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human fluid or derivative or preparation thereof, such as human serum or amniotic fluid, which contains human protein. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human albumin at a concentration of at least 0.5% and up to 10%. In particular embodiments, the human albumin concentration is from about 0.5 to about 2%. The human albumin may come from a liquid or a dried (powder) form and includes, but is not limited to, recombinant human albumin, PLASBUMIN® normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium, STEMLINE® T-cell expansion medium or STEMLINE® II hematopoietic stem cell expansion medium, OPTI-PRO™ serum-free culture medium, or DMEM culture medium, with human albumin (for example, PLASBUMIN® normal human serum albumin) added up to concentrations of 10%.

Optionally, other factors are used. In one embodiment, epidermal growth factor (EGF) at a concentration of between 0-1 μg/mL is used. In a preferred embodiment, the EGF concentration is around 10-20 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation. All supplements are clinical grade.

### Generation of ST266

The AMP cells of the invention can be used to generate ST266. In one embodiment, the AMP cells are isolated as described herein and 10×10⁶ cells are seeded into T75 flasks containing between 5-30 mL culture medium, preferably between 10-25 mL culture medium, and most preferably about 10 mL culture medium. The cells are cultured until confluent, the medium is changed and in one embodiment the ST266 is collected 1 day post-confluence. In another embodiment the medium is changed and ST266 is collected 2 days post-confluence. In another embodiment the medium is changed and ST266 is collected 3 days post-confluence. In another embodiment the medium is changed and ST266 is collected 4 days post-confluence. In another embodiment the medium is changed and ST266 is collected 5 days post-confluence. In another embodiment the medium is changed and ST266 is collected 3 days post-confluence. In another preferred embodiment the medium is changed and ST266 is collected 3, 4, 5, 6 or more days post-confluence. Skilled artisans will recognize that other embodiments for collecting ST266 from AMP cell cultures, such as using other tissue culture vessels, including but not limited to cell factories, bioreactors, flasks, hollow fibers, or suspension culture apparatus, or collecting ST266 from sub-confluent and/or actively proliferating cultures, are also contemplated by the methods of the invention. It is also contemplated by the instant invention that the ST266 be cryopreserved following collection. It is also contemplated by the invention that ST266 be lyophilized following collection. It is also contemplated that ST266 be formulated for sustained-release after collection. It is also contemplated that ST266 be formulated for targeted intranasal administration.

The compositions of the invention can be prepared in a variety of ways depending on the intended use of the compositions. For example, a composition useful in practicing the invention may be a liquid comprising an agent of the invention, i.e., ST266 and ACCS-N, and cells such as AMP cells and AMP-N cells compositions, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Pharmaceutical Compositions—The present invention provides pharmaceutical compositions of ST266 and/or AMP cells, and/or ACCS-N and/or AMP-N cell compositions and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits—The invention also provides for an article of manufacture comprising packaging material and a pharmaceutical composition of the invention contained within the packaging material, wherein the pharmaceutical composition comprises compositions of ST266 and/or AMP cells, and/or ACCS-N and/or AMP-N cell compositions. The packaging material comprises a label or package insert which indicates that the ST266 and/or AMP cells, and/or ACCS-N and/or AMP-N cell compositions can be used for targeted intranasal administration to treat ophthalmic disorders, diseases and injuries.

### Formulation, Dosage and Administration

Compositions comprising ST266 and/or AMP cells, and/or ACCS-N and/or AMP-N cells may be delivered by targeted intranasal administration to a subject to provide various cellular or tissue functions, for example, to treat ophthalmic disorders, diseases and injuries due to trauma, surgery, genetics, disease, inflammation, etc. As used herein “subject” may mean either a human or non-human animal.

Such compositions may be formulated for targeted intranasal administration in any conventional manner using one or more physiologically acceptable carriers optionally comprising excipients and auxiliaries. The compositions may be packaged with written instructions for their use in treating ophthalmic disorders, diseases and injuries. The compositions may also be delivered by targeted intranasal administration to the recipient in one or more physiologically acceptable carriers. Carriers for the cells may include but are not limited to solutions of phosphate buffered saline (PBS) or lactated Ringer's solution containing a mixture of salts in physiologic concentrations and the like.

Pharmaceutical compositions useful in the practice of the invention include a therapeutically effective amount of an active agent with a pharmaceutically acceptable carrier. Such pharmaceutical compositions may be liquid, gel, ointment, salve, slow release formulations or other formulations suitable for ophthalmic indications. The composition comprises a composition of the invention (i.e., ST266 and/or AMP cells, and/or ACCS-N and/or AMP-N cell) and, optionally, at least one ophthalmically acceptable excipient, for example, wherein the excipient is able to reduce a rate of removal of the composition from the front of the eye by lacrimation, such that the composition has an effective residence time on the eye of about 2 hours to about 24 hours or longer.

In various embodiments, compositions of the invention can comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

Aqueous compositions of the invention have ophthalmically compatible pH and osmolality. Optionally these compositions incorporate means to inhibit microbial growth, for example through preparation and packaging under sterile conditions and/or through inclusion of an antimicrobially effective amount of an ophthalmically acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

One of skill in the art may readily determine the appropriate concentration, or dose, of the ST266 and/or AMP cells, and/or ACCS-N and/or AMP-N cell compositions, for a particular purpose. The skilled artisan will recognize that a preferred dose is one which produces a therapeutic effect, such as treating and ophthalmic disorder, disease or injury, in a patient in need thereof. Of course, proper doses of the ST266 and/or AMP cells, and/or ACCS-N and/or AMP-N cell, will require empirical determination at time of use based on several variables including but not limited to the severity and type of disease, injury, disorder or condition being treated; patient age, weight, sex, health; other medications and treatments being administered to the patient; and the like. For example the compositions of the invention can be administered by targeted intranasal delivery as a solution (ST266 and/or AMP cells, and/or ACCS-N and/or AMP-N cells) or as a lyophilized or sprayed dried powder (ST266 and/or ACCS-N). In one embodiment, a targeted intranasal solution can be administered in varying volumes of 1 microliter to 2000 microliters of the ST266 and/or AMP cells, and/or ACCS-N and/or AMP-N cells compositions or as a 1 mg to 2000 mg of lyophilized or sprayed dried powder for ST266 and/or ACCS-N:. Each volume aliquot of the ST266 and/or AMP cells, and/or ACCS-N and/or AMP-N cell product dosage form can be administered to one or both nares of the subject using a device specifically suited to targeting the cribriform plate and olfactory filaments protruding from the olfactory bulb at the superior aspect of the nasal cavity. In order to achieve maximum bioavailability of the ST266 and/or AMP cells, and/or ACCS-N and/or AMP-N cells compositions, optimizing the dose volume or mass to achieve saturation concentrations in the olfactory nerve, and ultimately the optic nerve or the vitreous of the ocular globe. The ST266 and/or AMP cells, and/or ACCS-N and/or AMP-N cells intranasal dosage form can be administered one or more times per day dependent on the effective therapeutic dose needed to achieve the desired biological endpoint for the individual condition or patient being treated. In one embodiment, one dose is sufficient. Other embodiments contemplate 2, 3, 4, or more doses.

The present invention provides a method of treating ophthalmic disorders, disease and injuries by targeted intranasal administration to a subject ST266 and/or AMP cells, and/or ACCS-N and/or AMP-N cell compositions, in a therapeutically effective amount. By “therapeutically effective amount” is meant the dose of ST266 and/or AMP cells, and/or ACCS-N and/or AMP-N cell compositions, which is sufficient to elicit a therapeutic effect. Thus, the concentration of ST266 and/or AMP cells, and/or ACCS-N and/or AMP-N cell compositions in an administered dose unit in accordance with the present invention is effective in, for example, the treatment of disorders, disease and injuries. Applicant has shown that when ST266 is administered by targeted intranasal delivery it is found in the optic nerve, the optic chiasm, and globe of the eye, the caudate putamen, the cerebellum, the entorhinal cortex, the prefrontal cortex, the hippocampus, the olfactory bulb, the olfactory nerve, the substantia nigra, the trigeminal nerve, the trochlear nerve and vitreous of the eye as well as and other brain tissues. Thus, ST266 delivered in this fashion could be used to treat inflammation, disease and other cell-based dysfunctions of these tissues.

In further embodiments of the present invention, at least one additional neuroprotective agent may be combined with the ST266 and/or AMP cells, and/or ACCS-N and/or AMP-N cell compositions, to enhance neuroprotection of retinal cells, oligodendrocytes, Schwann cells, astrocytes etc. Such agents include, for example, antioxidants, such as, ascorbate, dimethylthiourea, α-tocopherol and (3-carotene; calcium antagonists, such as, flunarizine; growth factors, such as, basic-FGF, BDNF, CNTF, and IL-β; glucocorticoids such as methylprednisolone, dexamethasone; and iron chelators such as desferrioxamine. In addition, it may be desirable to co-administer other agents, including active agents and/or inactive agents, with the ST266 and/or AMP cells, and/or ACCS-N and/or AMP-N cell compositions, either for treating retinal diseases/disorders or to treat corneal diseases/disorders/injuries. Active agents include but are not limited to cytokines, chemokines, antibodies, inhibitors, antibiotics, anti-fungals, anti-virals, immunosuppressive agents, other cell types, and the like. Inactive agents include carriers, diluents, stabilizers, gelling agents, delivery vehicles, ECMs (natural and synthetic), scaffolds, and the like. When the ST266 and/or AMP cells, and/or ACCS-N and/or AMP-N cell compositions, are administered conjointly with other pharmaceutically active agents, (i.e., other neuroprotective agents) even less of the ST266 and/or AMP cells, and/or ACCS-N and/or AMP-N cell compositions, may be needed to be therapeutically effective.

In a preferred embodiment, ST266 and/or AMP cells, and/or ACCS-N and/or AMP-N cell compositions, are delivered by targeted intranasal administration to the nasal mucosa which is adjacent to the foramina of the cribriform plate located at the superior aspect of the nasal cavity, preferably via a delivery device suitable for targeted delivery to a specific location in the nasal cavity.

The timing of administration of ST266 and/or AMP cells, and/or ACCS-N and/or AMP-N cell compositions will depend upon the type and severity of the ophthalmic disorder being treated. In a preferred embodiment, the ST266 and/or AMP cells, and/or ACCS-N and/or AMP-N cell compositions, are administered as soon as possible after the ophthalmic disorder is diagnosed. In other preferred embodiments, the ST266 and/or AMP cells, and/or ACCS-N and/or AMP-N cell compositions, are administered more than one time following diagnosis.

Also contemplated by the methods of the invention are compositions comprising cells that have been partially or fully differentiated from AMP cells. Such partially or fully differentiated cell compositions are obtained by treating AMP cells with appropriate reagents and under appropriate conditions wherein the cells undergo partial or complete differentiation into, for example, retinal cells (i.e., rods cells and/or cones cells), retinal ganglion cells, limbal stem cells or corneal epithelial cells. Skilled artisans are familiar with conditions capable of effecting such partial or complete differentiation. The cells may be treated under differentiating conditions prior to targeted intranasal administration.

### Aerosol Compositions

Methods for creating aerosol compositions are well known to skilled artisans. Specifics can be found in “Development of Nasal Delivery Systems: A Review” By Jack Aurora in Drug Delivery and Development, volume 2, number 7, 2002, and “Drug Delivery to the Lung” By Hans Bisgaard, Christopher O'Callaghan, Gerald C. Smaldone, published by Informa Health Care, 2001, and elsewhere in the scientific literature. Such methods are useful in creating aerosol compositions of ST266 and/or AMP cells, and/or ACCS-N and/or AMP-N cells.

A “therapeutically effective amount” of a therapeutic agent within the meaning of the present invention will be determined by a patient's attending physician or veterinarian. Such amounts are readily ascertained by one of ordinary skill in the art and will enable treating ophthalmic disorders, diseases and injuries when administered by targeted intranasal administration in accordance with the present invention. Factors which influence what a therapeutically effective amount will be include, the specific activity of the therapeutic agent being used, the condition being treated, the absence or presence of infection, time elapsed since diagnosis or injury, and the age, physical condition, existence of other disease states, and nutritional status of the patient. Additionally, other medication the patient may be receiving will effect the determination of the therapeutically effective amount of the therapeutic agent to administer.

The treatment of ophthalmic disorders, diseases and injuries by targeted intranasal administration of therapeutic agents can be monitored by employing a variety of tests and measurements including but not limited to standard visual acuity tests, the Amsler Grid Test, fluorescein angiography, optical coherence tomography, and ERG.

### Exemplary Therapeutic Uses

### Disorders/Diseases/Injuries of the Cornea

Keratitis refers to inflammation of the cornea. Causes include but are not limited to amoebic, bacterial, fungal or viral infection, photokeratitis, exposure (eyelid dysfunction), chemical injury, trauma, surgery (LASIK, PRK, cataract, corneal transplant, pterygium surgery), or congenital causes such as keratoconus, Fuchs' dystrophy, or keratoconjunctivitis sicca. The method of targeted intranasal administration of the ST266 and/or AMP cells, and/or ACCS-N and/or AMP-N cell compositions of the present invention may be used to treat keratitis.

Corneal ulcers form when the surface of the cornea is damaged or compromised in some way. The ulcers may be sterile or infected and determines the course of treatment. Bacterially infected ulcers tend to be extremely painful and are typically associated with a break in the corneal epithelium, the outermost layer of the cornea. Certain types of bacteria, such as Pseudomonas, are extremely aggressive and can cause severe damage and even blindness within 24-48 hours if left untreated. Sterile ulcers cause little if any pain. They are often found near the peripheral edge of the cornea and are not necessarily accompanied by a break in the corneal epithelium. There are many causes of corneal ulcers. Contact lens wearers are at an increased risk of corneal ulcers if they are not diligent in the cleaning, handling, and disinfection of their lenses and lens cases. Bacterially infected ulcers are also associated with diseases that compromise the corneal surface, creating a window of opportunity for organisms to infect the cornea. Patients with severely dry eyes, who have difficulty blinking, or who are unable to care for themselves, are also at risk. Other causes of ulcers include herpes simplex viral infections, inflammatory diseases, corneal abrasions or injuries, and other systemic diseases. The method of targeted intranasal administration of the ST266 and/or AMP cells, and/or ACCS-N and/or AMP-N cell compositions of the present invention may be used to treat corneal ulcers.

Corneal wounds are injuries to the ocular surface and can be thermal wounds (i.e., burns), chemical wounds (i.e., acids), physical wounds (i.e., abrasions), surgical wounds (i.e., corneal transplant), or a combination of these wound types. The method of targeted intranasal administration of the ST266 and/or AMP cells, and/or ACCS-N and/or AMP-N cell compositions of the present invention may be used to treat corneal wounds.

Dry eye syndrome is one of the most common problems treated by ophthalmologists. It is usually caused by a problem with the quality of the tear film that lubricates the eyes. Tears are comprised of three layers. The inner mucus layer coats the cornea, forming a foundation so the tear film can adhere to the eye, the middle aqueous layer provides moisture and supplies oxygen and other important nutrients to the cornea, and the outer lipid layer is an oily film that seals the tear film on the eye and helps to prevent evaporation. Tears are formed by several glands around the eye. The middle aqueous layer is produced in the lacriminal gland located under the upper eyelid and several smaller glands in the lids make the outer lipid and inner mucus layers. With each blink, the eyelids spread the tears over the eye surface. Excess tears flow into two tiny drainage ducts in the corner of the eye by the nose. These ducts lead to tiny canals that connect to the nasal passage. Dry eye syndrome has many causes. One of the most common reasons for dryness is the normal aging process. Many other factors, such as hot, dry or windy climates, high altitudes, air-conditioning and cigarette smoke also cause dry eyes. Many people also find their eyes become irritated when reading or working on a computer. Contact lens wearers may also suffer from dryness because the contacts absorb the tear film, causing proteins to adhere to the surface of the contact lens. Certain medications, thyroid conditions, vitamin A deficiency, menopause and diseases such as Parkinson's and Sjogren's syndrome can also cause dryness. The method of targeted intranasal administration of the ST266 and/or AMP cells, and/or ACCS-N and/or AMP-N cell compositions of the present invention may be used to treat dry eye syndrome.

Sjogren's syndrome is a disorder of the immune system identified by its two most common symptoms—dry eyes and a dry mouth. Sjogren's syndrome often accompanies other immune system disorders, such as rheumatoid arthritis and lupus. In Sjogren's syndrome, the mucous membranes and moisture-secreting glands of your eyes and mouth are usually affected first, resulting in decreased production of tears and saliva. Although Sjogren's syndrome can develope at any age, most people are older than 40 at the time of diagnosis. The condition is much more common in women. Current treatment focuses on relieving symptoms. The method of targeted intranasal administration of the ST266 and/or AMP cells, and/or ACCS-N and/or AMP-N cell compositions of the present invention may be used to treat dry eye associated with Sjogren's syndrome.

Allergic conjunctivitis occurs when the conjunctiva becomes swollen or inflamed due to a reaction to pollen, dander, mold, or other allergy-causing substances. The conjunctiva is a clear layer of tissue lining the eyelids and covering the white of the eye. When the eyes are exposed to allergy-causing substances, a substance called histamine is released by the body. The blood vessels in the conjunctiva become enlarged and the eyes can become red, itchy, and teary very quickly. The pollens that cause symptoms vary from person to person and from area to area but generally include pollen from grasses, ragweed and trees. Symptoms may be seasonal and can include intense itching or burning eyes, puffy eyelids, especially in the morning, red eyes, stringy eye discharge, tearing, dilated blood vessels in the clear conjunctival tissue covering the white of the eye The method of targeted intranasal administration of the ST266 and/or AMP cells, and/or ACCS-N and/or AMP-N cell compositions of the present invention may be used to treat allergic conjunctivitis.

Corneal transplantation is surgery to replace the cornea with tissue from a deceased donor. It is one of the most common transplants done. The donated cornea is processed and tested by a local eye bank to make sure it is safe for use in your surgery. The most common type of corneal transplant is called penetrating keratoplasty. During this procedure, the surgeon removes a small round piece of the cornea. The donated tissue will then be sewed into the surgically created opening. A newer technique is called lamellar keratoplasty. In this procedure, only the inner or outer layers of the cornea are replaced, rather than all of the layers. This technique can lead to faster recovery and fewer complications. A corneal transplant is recommended for people who have vision problems caused by thinning of the cornea, most often due to keratoconus, scarring of the cornea from severe infections or injuries, vision loss caused by cloudiness of the cornea, most often due to Fuchs' dystrophy. The body may reject the transplanted tissue. This occurs in about one out of three patients in the first 5 years. Rejection can sometimes be controlled with steroid eye drops. The method of targeted intranasal administration of the ST266 and/or AMP cells, and/or ACCS-N and/or AMP-N cell compositions of the present invention may be used to prevent corneal transplant rejection.

### Disorders/Diseases/Injuries of the Retina

Macular holes (also called macular cysts, retinal holes, retinal tears, and retinal perforations) may occur for a variety of reasons, but are usually a result of traction from the vitreous gel on the macula. Since the macula is responsible for central vision, this problem causes severe and often complete loss of central vision. It is possible for anyone to develop a macular hole, but they are most common among women about 60-70 years of age. Macular holes are typically treated with a surgical technique called transpars plana vitrectomy, which removes the vitreous and replaces it with an air/gas bubble to hold the retina in place while the hole is repaired. Eventually, the body replaces the air/gas bubble with natural fluids. Unfortunately, the surgery itself may permanently damage central vision. Current methods for treating macular holes improve vision in only 40% of eyes. The method of targeted intranasal administration of the ST266 and/or AMP cells, and/or ACCS-N and/or AMP-N cell compositions of the present invention may be used to treat macular holes.

Retinal detachment occurs when the retina's sensory and pigment layers separate. Because it can cause devastating damage to the vision if left untreated, retinal detachment is considered an ocular emergency that requires immediate medical attention and surgery. There are three types of retinal detachments. The most common type occurs when there is a break in the sensory layer of the retina, and fluid seeps underneath, causing the layers of the retina to separate. The second most common type occurs when strands of vitreous or scar tissue create traction on the retina, pulling it loose. Patients with diabetes are more likely to experience this type. The third type happens when fluid collects underneath the layers of the retina, causing it to separate from the back wall of the eye. This type usually occurs in conjunction with another disease affecting the eye that causes swelling or bleeding. The method of targeted intranasal administration of the ST266 and/or AMP cells, and/or ACCS-N and/or AMP-N cell compositions of the present invention may be used to treat retinal detachment.

Retinal degeneration occurs when the photoreceptor cells (rods and cones) are progressively lost due to disease or injury. There are many types of retinal degeneration including Age-Related Macular Degeneration (AMD), which can be either the more common “dry” form or the less common, but more serious, “wet” form. Stargardt disease is an inherited juvenile macular degeneration disorder. Dry AMD cannot be cured, but patients with the condition should continue to remain under an ophthalmologist's care to monitor the affected eye. Also, if the other eye is healthy, screening still should continue, to stay on the lookout for problems. Wet AMD may be successfully treated with laser surgery. However, successful treatment may not mean restoring normal vision, but rather, preventing vision loss from worsening. One drawback of laser surgery is that it may damage some of the neighboring retinal tissue. There are several surgical procedures that may be used depending on the size and type of the abnormal blood vessels. One surgical procedure, called laser photocoagulation, destroys leaking blood vessels that have grown under the macula and halts the damage. A newer laser procedure called photodynamic therapy uses a different laser to treat abnormal blood vessels and a medication injected into the patient's arm. This medication travels through the bloodstream and attaches itself to the abnormal blood vessels, so when the laser light is shown in the eye, the blood vessels alone are destroyed. Both of these procedures must be done before the abnormal blood vessels leak and cause irreversible damage to the retina. Also, because more blood vessels could grow later on, patients who get this treatment need to continue to have follow-up evaluations. In addition to surgery, several new drugs are on the market or in development to treat macular degeneration. These include VEGF inhibitors such as EYLEA® (Regeneron Pharmaceuticals, Inc.) and other types of molecules. The method of targeted intranasal administration of the ST266 and/or AMP cells, and/or ACCS-N and/or AMP-N cell compositions of the present invention may be used to treat retinal degeneration, including macular degeneration.

Retinitis Pigmentosa (RP) refers to a group of inherited retinal degeneration disorders. The most common feature of all forms of RP is the gradual degeneration of the rods and cones. Most forms of RP first cause the degeneration of rod cells. These forms of RP, sometimes called rod-cone dystrophy, usually begin with night blindness. Patients with RP cannot adjust well to dark and dimly lit environments. As the disease progresses and more rod cells degenerate, patients lose their peripheral vision. Patients with RP often experience a ring of vision loss in their mid-periphery with small islands of vision in their very far periphery. Others report the sensation of tunnel vision, as though they see the world through a straw. Many patients with RP retain a small degree of central vision throughout their life. Usher syndrome is a type of RP that is also associated with hearing loss. Unfortunately, no clinically significant treatment currently exists for RP, although much research in the field of gene therapy and stem cell therapy is underway. The method of targeted intranasal administration of the ST266 and/or AMP cells, and/or ACCS-N and/or AMP-N cell compositions of the present invention may be used to treat RP.

Light-induced retinal degeneration includes, but is not limited to, medical-light induced retinal degeneration. Some RP patients are more sensitive to light damage than others (see Paskowitz, D. M., et al., (Br J Ophthalmol 2006;90:1060-1066). Protecting such patients by targeted intranasal administration of the compositions of the invention prior to medical invention utilizing potentially damaging light is contemplated by the novel methods of the invention.

Choroideremia is a rare inherited disorder that causes progressive loss of vision due to degeneration of the choroid and retina. Formerly called tapetochoroidal dystrophy, choroideremia occurs almost exclusively in males. In childhood, night blindness is the most common first symptom. As the disease progresses, there is loss of peripheral vision or “tunnel vision”, and later a loss of central vision. Progression of the disease continues throughout the individual's life, although both the rate and the degree of visual loss can vary, even within the same family. Vision loss due to choroideremia is caused by degeneration of several layers of cells that are essential to sight. These layers, which line the inside of the back of the eye, are called the choroids, the retinal pigment epithelium and the photoreceptors. The retinal pigment epithelium and the choroid initially deteriorate to cause choroideremia. Eventually, the photoreceptors break down as well. The method of targeted intranasal administration of the ST266 and/or AMP cells, and/or ACCS-N and/or AMP-N cell compositions of the present invention may be used to treat choroideremia.

Retinoschisis is a rare eye disorder characterized by the abnormal splitting of the retina's sensory layers, resulting in loss of visual function. It is estimated that retinoschisis affects one in 5,000 to 25,000 individuals, primarily young males. Treatment is often aimed at restricting any worsening of the separation so that it does not encroach on the macula. Retinoschisis causes acuity loss in the center of the visual field through the formation of tiny cysts in the retina. The cysts are usually only detectable by a trained clinician. Vision cannot be improved by corrective lenses, as the nerve tissue itself is damaged by these cysts. The method of targeted intranasal administration of the ST266 and/or AMP cells, and/or ACCS-N and/or AMP-N cell compositions of the present invention may be used to treat retinoschisis.

Diabetic retinopathy occurs as a complication of diabetes. Types of diabetic retinopathy include background diabetic retinopathy, pre-proliferative diabetic retinopathy, clinically significant diabetic macular edema and proliferative diabetic retinopathy. Diabetic retinopathy is characterized by vitreous or retinal hemorrhage, retinal microaneurysm, retinal neovascularization and macular edema. During the first three stages of diabetic retinopathy, no treatment is needed, unless macular edema is present. To prevent progression of diabetic retinopathy, diabetics should control their levels of blood sugar, blood pressure, and blood cholesterol. Proliferative retinopathy is treated with laser surgery called scatter laser treatment. Scatter laser treatment helps to shrink the abnormal blood vessels. Because a high number of laser burns are necessary, two or more sessions usually are required to complete treatment. Scatter laser treatment works better before the fragile, new blood vessels have started to bleed. However, even if bleeding has started, scatter laser treatment may still be possible, depending on the amount of bleeding. If the bleeding is severe, patients may need a surgical procedure called a vitrectomy. During a vitrectomy, blood is removed from the center of the eye. The method of targeted intranasal administration of the ST266 and/or AMP cells, and/or ACCS-N and/or AMP-N cell compositions of the present invention may be used to treat diabetic retinopathy.

Retinal ischemia occurs when there is a lack of oxygen to the cells of the retina and results in damage or death the retinal cells and consequent loss of vision. Causes include various retinal vascular disorders such as retinal venous occlusion. Hypertension is a risk factor for retinal ischemia. The method of targeted intranasal administration of the ST266 and/or AMP cells, and/or ACCS-N and/or AMP-N cell compositions of the present invention may be used to treat retinal ischemia.

Retinopathy of Prematurity (ROP), previously known as retrolental fibroplasia, is a disease of the eye that affects premature babies. It is thought to be caused by the disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP. The method of targeted intranasal administration of the ST266 and/or AMP cells, and/or ACCS-N and/or AMP-N cell compositions of the present invention may be used to treat ROP.

Retinal transplantation. The method of targeted intranasal administration of the AMP cell and/or AMP-N cell compositions of the present invention may be used to treat preventrejection of transplanted retinal tissue. Briefly, it has been discovered that AMP cells alone or in combination with other suitable active agents, are useful agents capable of treating HVG, GVHD, as well as many other immune diseases and disorders (see, for example, U.S. Published Application No. 2010-0068180-A1, which is incorporated herein in its entirety). The cells express HLA-G, do not express MHC Class II antigens, are telomerase negative, do not form teratomas, are not immortal, secrete cellular modulatory factors, and are readily available in great numbers.

Diseases, Disorders and Injuries of the Optic Nerve

Optic Neuritis is a demyelinating inflammation of the optic nerve. It is also known as optic papillitis (when the head of the optic nerve is involved) and retrobulbar neuritis (when the posterior of the nerve is involved). It is often observed as one of the early symptoms of multiple sclerosis, and it may lead to complete or partial loss of vision in one or both eyes. The method of targeted intranasal administration of the ST266 and/or AMP cells, and/or ACCS-N and/or AMP-N cell compositions of the present invention may be used to treat optic neuritis.

Optic neuropathy is a term that refers to damage to the optic nerve regardless of the cause. Damage and death of the neurons leads to the characteristic features of optic neuropathy including loss of vision and colors appearing subtly washed out in the affected eye. On medical examination, the optic nerve head can be visualized by an ophthalmoscope. A pale disc is characteristic of long-standing optic neuropathy. In many cases, only one eye is affected and the patient may not be aware of the loss of color vision until the ophthalmologist asks him to cover the unaffected eye. The method of targeted intranasal administration of the ST266 and/or AMP cells, and/or ACCS-N and/or AMP-N cell compositions of the present invention may be used to treat optic neuropathy.

Non-arteritic anterior ischemic optic neuropathy (NAION) refers to loss of blood flow to the optic nerve. This condition typically causes sudden vision loss in one eye, without any pain. In many cases, the patient notices significant loss of vision in one eye immediately upon waking up in the morning. The visual loss typically remains fairly stable, without getting markedly better or worse once it has occurred. The method of targeted intranasal administration of the ST266 and/or AMP cells, and/or ACCS-N and/or AMP-N cell compositions of the present invention may be used to treat non-arteritic anterior ischemic optic neuropathy.

Arteritic anterior ischemic optic neuropathy (AION) is associated with giant cell arteritis (GCA; often termed temporal arteritis). AION is characterized by visual loss associated with optic disc swelling, sometimes with flame hemorrhages on the swollen disc or nearby neuro-retinal layer, and sometimes with nearby cotton-wool exudates. Visual loss is usually sudden or develops over a few days at most and is commonly unilateral, although second eye involvement may occur later. The visual loss is usually permanent, with some recovery possibly occurring within the first weeks or months. The method of targeted intranasal administration of the ST266 and/or AMP cells, and/or ACCS-N and/or AMP-N cell compositions of the present invention may be used to treat Arteritic anterior ischemic optic neuropathy.

Traumatic optic neuropathy (TON) refers to an acute injury of the optic nerve secondary to trauma. The optic nerve axons may be damaged either directly or indirectly and the visual loss may be partial or complete. An indirect injury to the optic nerve typically occurs from the transmission of forces to the optic canal from blunt head trauma. This is in contrast to direct TON, which results from an anatomical disruption of the optic nerve fibers from penetrating orbital trauma, bone fragments within the optic canal, or nerve sheath hematomas. The method of targeted intranasal administration of the ST266 and/or AMP cells, and/or ACCS-N and/or AMP-N cell compositions of the present invention may be used to treat traumatic optic neuropathy.

Leber's optic neuropathy (LHON) or Leber optic atrophy is a mitochondrially inherited degeneration of retinal ganglion cells (RGCs) and their axons that leads to an acute or subacute loss of central vision and affects predominantly young adult males. LHON is only transmitted through the mother, as it is primarily due to mutations in the mitochondrial (not nuclear) genome, and only the oocyte contributes mitochondria to the embryo. The method of targeted intranasal administration of the ST266 and/or AMP cells, and/or ACCS-N and/or AMP-N cell compositions of the present invention may be used to treat Leber's optic neuropathy.

Dominant optic atrophy, or dominant optic atrophy, Kjer's type, is an autosomally inherited disease that affects the optic nerves, causing reduced visual acuity and blindness beginning in childhood. This condition is due to mitochondrial dysfunction mediating the death of optic nerve fibers. Although dominant optic atrophy is the most common autosomally inherited optic neuropathy aside from glaucoma, it is often misdiagnosed. The method of targeted intranasal administration of the ST266 and/or AMP cells, and/or ACCS-N and/or AMP-N cell compositions of the present invention may be used to treat dominant optic atrophy.

Recessive optic atrophy is a rare autosomal recessive disorder that leads to vision loss. The method of targeted intranasal administration of the ST266 and/or AMP cells, and/or ACCS-N and/or AMP-N cell compositions of the present invention may be used in treating optic neuritis.

Radiation-induced optic neuropathy (RION) is a devastating late complication of radiotherapy to the anterior visual pathway resulting in acute, profound, irreversible visual loss. It is thought to be a result of radiation necrosis of the anterior visual pathway. The method of targeted intranasal administration of the ST266 and/or AMP cells, and/or ACCS-N and/or AMP-N cell compositions of the present invention may be used to treat recessive optic atrophy.

Neuromyelitis optica spectrum disorder (NMOSD) is a recently proposed unifying term for neuromyelitis optica (NMO), also known as Devic's disease, and related syndromes. It is a relapsing inflammatory demyelinating disease that most commonly affects the optic nerves and the spinal cord, leading to sudden vision loss or weakness in one or both eyes, and loss of sensation and bladder function. The condition may also target other parts of the brain, especially the brainstem and hypothalamus, causing signs and symptoms such as severe and persistent vomiting and hiccups, or sleeping and eating disorders. Attacks of NMOSD tend to be more severe and often different in nature from those of the prototype form of multiple sclerosis (MS), another relapsing inflammatory disease of the optic nerves, spinal cord and brain; however, MS and NMOSD are often confused. The method of targeted intranasal administration of the ST266 and/or AMP cells, and/or ACCS-N and/or AMP-N cell compositions of the present invention may be used to treat NMOSD.

Optic Nerve Crush is a traumatic injury to the optic nerve that leads to retinal ganglion cell and glial cell death and potentially complete loss of vision. The method of targeted intranasal administration of the ST266 and/or AMP cells, and/or ACCS-N and/or AMP-N cell compositions of the present invention may be used in treating optic nerve crush.

Optic Nerve Blunt Force Trauma is traumatic injury to the optic nerve that leads to retinal ganglion cell and glial cell death and potentially complete loss of vision. The method of targeted intranasal administration of the ST266 and/or AMP cells, and/or ACCS-N and/or AMP-N cell compositions of the present invention may be used to treat optic nerve blunt force trauma.

Glaucoma is a group of eye conditions that damage the optic nerve. This damage is often caused by an abnormally high intraocular pressure. Glaucoma is one of the leading causes of blindness in the United States. It can occur at any age but is more common in older adults. The most common form of glaucoma has no warning signs. The effect is so gradual that a patient may not notice a change in vision until the condition is at an advanced stage. Vision loss due to glaucoma cannot be recovered. If glaucoma is diagnosed early, vision loss can be slowed or prevented. The method of targeted intranasal administration of the ST266 and/or AMP cells, and/or ACCS-N and/or AMP-N cell compositions of the present invention may be used to reduce damage to the optic nerve caused by the increased intraocular pressure seen in patients suffering from glaucoma.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Amnion epithelial cells were dissociated from starting amniotic membrane using dissociation agent. The average weight range of an amnion was 18-27 g. The number of cells recovered per g of amnion was about 10-15×10⁶.

Method of obtaining selected AMP cells—Amnion epithelial cells were either cryopreserved or plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in basal medium supplemented r human serum albumin until they reached 120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use.

### Example 2

### Generation of ST266

The AMP cells of the invention were used to generate ST266 as follows. A placenta was obtained and the amnion was isolated from the placenta, amnion epithelial cells were enzymatically released from the amnion, the released amnion-derived epithelial cells were collected, the collect cells were cultured in IMDM culture medium that was supplemented with 0.5% human serum albumin and 10 ng/mL recombinant human EGF. The culture medium was collected after about 2-3 days and fresh culture medium was applied. The collected of culture medium and application of fresh culture medium was repeated a plurality of times. It is contemplated by the instant invention that the ST266 be cryopreserved, lyophilized, irradiated, diluted, concentrated or formulated for sustained-release following collection.

### Example 3

### Intranasal Delivery of ¹²⁵I-labeled ST266

Model: ¹²⁵I-labeled ST266 was delivered by intranasal delivery to rats as described in Shyeilla V. Dhuria, Leah R. Hanson, and William H. Frey, II, Novel Vasoconstrictor Formulation to Enhance Intranasal Targeting of Neuropeptide Therapeutics to the Central Nervous System, The Journal Of Pharmacology And Experimental Therapeutics, 328:312-320, 2009.

Results: Significant quantities of ¹²⁵I-labeled ST266 delivered by intranasal delivery were deposited on the rat optic nerve (1000 ng ST266/g tissue) and in the vitreous (900 ng ST266/g tissue) as compared to blood (100 ng ST266/g tissue), olfactory bulb (50 ng ST266/g tissue) and trigeminal nerve (25 ng ST266/g tissue). Thus, intranasal delivery of ST266 and other therapeutic agents represents a novel and feasible approach to treat ophthalmic diseases, disorders and injuries.

### Example 4

### Neuroprotective Effects of ST266 in Experimental Optic Neuritis

Optic neuritis is a demyelinating inflammation of the optic nerve that often occurs in multiple sclerosis (MS) patients. Loss of retinal ganglion cells (RGCs) and their axons also occurs in optic neuritis, and correlates with permanent vision loss. ST266 is a novel biologic mixture of growth factors and cytokines secreted from AMP cells that exhibits anti-inflammatory and neuroprotective properties in a variety of disease models. The ability of ST266 to suppress optic neuritis in the experimental autoimmune encephalomyelitis (EAE) model of MS was examined.

Method: Experimental autoimmune encephalomyelitis (EAE) was induced by active immunization with the myelin oligodendrocyte glycoprotein (MOG) in C57/BL6 mice. Mice were placed in the supine position for administration of one drop (6 uL) of ST266 intranasally both at the time of MOG antigen immunization or starting on day 15 coinciding with the symptom optic neuritis onset. Visual function was assessed by optokinetic responses (OKR) at baseline, then weekly until sacrifice 6 weeks post-immunization. Retinas and optic nerves were isolated. Retinal Ganglion Cells (RGCs) were immunolabeled with Brn3a antibodies to quantify RGC survival. Inflammation was assessed by H&E and Ibal (macrophage/microglia marker) staining. Demyelination was assessed by luxol fast blue staining, and axonal loss was assessed by neurofilament staining of optic nerve sections.

Results: Progressive decreases in OKR occurred in vehicle-treated EAE mice, along with significant RGC loss, consistent with prior studies showing onset of optic neuritis occurring 12-15 days after EAE induction. Daily intranasal ST266 treatment beginning on day 0 (day of immunization), 15, 22, or 30, significantly reduced the level of vision loss, and treatment from day 0 or day 15 significantly attenuated RGC loss. ST266 also decreased the degree of demyelination and axonal loss, and reduced the level of inflammation in the optic nerve quantified by reduced Ibal immunostaining indicating reduced microglia nerve injury.

Conclusions: Intranasal delivery of ST266 attenuates RGC loss, preserves OKR responses, and reduces demyelination and axonal loss during experimental optic neuritis in EAE mice. ST266 exerts effects with treatment initiated before and after onset of optic neuritis, suggesting it may be useful as a preventative or abortive therapy. Results suggest ST266 is a potential treatment for optic neuritis. Furthermore, potent effects seen after intranasal administration suggest this may be a novel drug delivery method for optic neuritis.

### Example 5

### Neuroprotective Effects of ST266 in Experimental Optic Neuritis with Multiple Daily Intranasal Dosing

Method: Optic neuritis was induced in the MS model EAE by immunization of 8 week old female C57BL/6J mice with myelin antigen (MOG). Control mice were sham-immunized with PBS. Visual function was assessed by OKR weekly. EAE and control mice consisted of the following treatment groups: a) 4 control (non-EAE mice), b) 6 EAE mice—sham treated mice with intranasal PBS beginning day 15 post-immunization (disease onset), c) 6 EAE mice—sham treated mice with intranasal Human Serum Albumin (HAS) beginning day 15 post-immunization (disease onset), d) 6 EAE mice—treated daily with intranasal ST266 beginning day 15, and continuing for 2 weeks, then treated with PBS until sacrifice at day 56,be) 6 EAE mice—treated twice daily with intranasal ST266 beginning day 15, and continuing for 2 weeks, then treated with PBS until sacrifice at day 56, f) 6 EAE mice—treated daily with intranasal ST266 beginning day 15, until sacrifice on day 56, g) 6 EAE mice—treated twice daily with intranasal ST266 beginning day 15, until sacrifice on day 56.

Results: Group a) control mice maintained consistent OKR scores with no loss of visual acuity. Groups b) and c) mice treated with PBS or HSA showed continual loss of visual acuity by OKR commencing on day 15 and progressing throughout the 56 day experiment. Group d) and e) mice treated only for days 15 through 30 showed improvement in visual acuity coincident with ST266 treatment, however the protective targeted neural effect was not maintained after intranasal PBS from days 30 to 56 was substituted for ST266. There was no difference in mice treated once or twice daily.

Retinal Ganglion Cell survival cell by labeling and cell counting revealed that ST266 treated mice showed significant increases in RCG survival compared to PBS and HSA treated mice.

Groups f) and g) showed comparable significant improvement in visual acuity independent of whether ST266 was intranasally administered once or twice per day.

Conclusions: Continuous ST266 treatment prevented vision loss. Once daily intranasal ST266 was as effective as twice daily ST266. The ST266 effect was not maintained 1-2 weeks after treatment was suspended. Intranasal HSA had no independent effect. Continuous ST266 treatment prevented RGC loss measured at day 56. Twice daily intranasal ST266 maintained RCGs comparable to daily ST266. Maintenance of RCG survival in animals ceased in groups treated once daily with intranasal ST266 after treatment stopped. There was an observable protective effect in RCG survival in animals treated twice daily with ST266 even after treatment was stopped. HSA had no independent effect on RCG survival.

### Example 6

### Blinded Sample Neuroprotective Effects of Intranasal ST266 in Experimental Optic Neuritis Compared with Control Cell Growth Media

Optic neuritis was induced in the MS model EAE by immunization of 8-week old female C57BL/6J mice with myelin antigen (MOG). The intranasal treatment solutions were blinded and labeled A, B and C. Visual function was assessed by OKR weekly. Six mice in each group were treated daily with intranasal administration of 6 μL of either solution A, B or C. The treatment dosage form solutions were revealed to the blinded investigators only upon completion of the experiment. A control group received no MOG antigen served as a positive control.

Results: Group A showed continuous loss of visual acuity from day 15 until the end of the experiment. Group B showed loss of visual acuity at day 15 that recovered to non-immunized control groups by day 42. Group C showed continuous loss of visual acuity from day 15 until the end of the experiment. Un-blinding the groups revealed that Group A was PBS, Group B was ST266 and Group C was STM100. Retinal Ganglion Cell number was significantly increased in Group B.

Conclusions: Only the intranasal treated ST266 group showed recovery of visual acuity and significant increases in RCGs. Groups A and C lost visual acuity in a manner to untreated MOG immunized mice. This study independently supported the action of intranasal ST266 to treat the loss of visual in a chronic model of optic neuritis. No effect in visual acuity or RCG survival was observed using the growth media used in the production of ST266.

### Example 7

### Evaluation of the Distribution of Targeted Intranasally Delivered I-¹²⁵ Radiolabeled ST266 in a Non-Human Primate Animal Model

The purpose of this study was to evaluate the distribution of targeted intranasally delivered I-¹²⁵ radiolabeled ST266 in a non-human primate animal model.

Methods: Human serum albumin-free ST266 was radiolabeled with Iodine-¹²⁵. Eight cynomolgus monkeys (males, ˜3.5 kg) where distributed into the following treatment groups: Group 1—Control solution/Evans Blue Dye (n=2) delivered using a targeted intranasal delivery device; Group 2—I-¹²⁵ ST266 (n=3) delivered intranasally using a gavage tube/syringe; and Group 3—I-¹²⁵ ST266 (n=3) delivered using a targeted intranasal delivery device.

The animals were anesthetized with sodium pentobarbital and given 4×125 μl doses per each nare with the treatment agent as indicated above. Each treatment was designed to target the cribriform plate located at the superior aspect of the nasal cavity and the olfactory bulb. The animals were euthanized with sodium pentobarbital and at the following time points and samples were collected from the brain, ocular tissues, stomach, and lungs. Autoradiography was also performed (at what point? I assume after euthanasia but before sample collection)

Results

Ocular tissues: Evans Blue Dye was visually detected in the olfactory bulb tract, along the olfactory bulb and surrounding the eye socket. I-¹²⁵ ST266 deposition was observed in the olfactory nerve tract, optic nerve, and vitreous. SDS PAGE analysis indicated the presence of low, medium and high molecular weight material in optic nerve and vitreous. It was observed that increased incubation time yielded greater optic nerve and vitreous deposition.

Brain tissues: Targeted intranasal delivery resulted in significant deposition of I-¹²⁵ radiolabeled ST266 on numerous right and left side brain tissues including the caudate putamen, the cerebellum, the entorhinal cortex, the prefrontal cortex, the hippocampus, the olfactory bulb, the olfactory nerve, the substantia nigra, the trigeminal nerve, the trochlear nerve, the optic nerve, the optic chiasm, and globe of the eye, and the vitreous humor. This study clearly showed the ability to deliver large molecular proteins to the brain via targeted intranasal administration.

### Example 8

### Traumatic Optic Neuropathy Animal Model

Traumatic optic neuropathy was modeled in rodents by crushing the nerve with forceps, resulting in loss of vision and degeneration of retinal ganglion cells (RGCs) (see, for example, Zuo, et al., “SIRT1 promotes RGC survival and delays loss of function following optic nerve crush”, Invest Ophthalmol Vis Sci 54(7):5097-5102, 2013)). RGC function was measured by pupillometry and optokinetic responses, and RGC survival was quantified, showing that this model provides a unique opportunity to assess neuroprotective therapies for traumatic CNS injuries.

The animals were treated with intranasal delivery of ST266. Optic nerve inflammation, demyelination and axonal injury were assessed by histologic and immunohistochemical staining of optic nerve sections as in prior studies (Shindler, et al., “Inflammatory demyelination induces axonal injury and retinal ganglion cell apoptosis in experimental optic neuritis”, Exp Eye Res 87(3):208-213, 2008). Terminal deoxynucleotidyl transferase-mediated biotinylated UTP nick end labeling (TUNEL), a marker of apoptosis, is used to identify dying RGCs. OKR measurements measured over 5 days following optic nerve crush showed significant improvement in visual acuity upon treatment with targeted intranasal administration of ST266. Optic nerve tissues showed greater retinal ganglion cell number and neuronal survival after ST266 intranasal administration. These animals also showed reduced optic nerve inflammation, and reduced axonal loss in the ST266 treated group.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A method for delivering a therapeutic agent to the eye in a patient in need thereof comprising targeted intranasal administration of the therapeutic agent to the patient.

2. The method of claim 1 wherein the therapeutic agent is a small molecular weight agent.

3. The method of claim 2 wherein the small molecular weight agent is a biological.

4. The method of claim 2 wherein the small molecular weight agent is a chemical.

5. The method of claim 2 wherein the small molecular weight agent has a molecular weight equal to or less than 900 daltons.

6. The method of claim 1 wherein the therapeutic agent is a large molecular weight agent.

7. The method of claim 6 wherein the large molecular weight agent is a biological.

8. The method of claim 6 wherein the large molecular weight agent is a chemical.

9. The method of claim 6 wherein the large molecular weight agent has a molecular weight greater than 900 daltons.

10. The method of claim 1 wherein the therapeutic agent is a complex biological composition.

11. The method of claim 10 wherein the complex biological composition is selected from the group consisting of ST266 and ACCS-N.

12. The method of claim 1 wherein the therapeutic agent is a population of cells.

13. The method of claim 12 wherein the population of cells is selected from the group consisting of AMP cells and AMP-N cells.

14. The method of claim 1 wherein the patient is afflicted with an ophthalmic disorder, disease or injury.

15. The method of claim 14 wherein the ophthalmic disorder, disease or injury is selected from the group consisting of a corneal disorder, disease or injury, a lens disorder, disease or injury, a retinal disorder, disease or injury and an optic nerve disorder, disease or injury.

16. The method of claim 1 wherein the therapeutic agent is administered in combination with other agents or treatment modalities.

17. The method of claim 16 wherein the other agents are active agents.
